(19) United States
(12) Patent Application Publication
(10) Pub. No.: US 2005/0257997 A1
(43) Pub. Date: Nov. 24, 2005

(54) Safescope caps

(76) Inventors: Vicky Larraine Vick, Memphis, TN (US); Robert Louis Vick JR., Memphis, TN (US)

Correspondence Address: Robert L. Vick Jr., 4282 Gray Fox Cu., Memphis, TN, 38141, US

(21) Appl. No.: 10/851,969

(22) Filed: May 24, 2004

(51) Int. Cl. A61B 7/02

(52) U.S. Cl. 181/131

(57) ABSTRACT

“SafeScope Caps” are a new revolutionary idea in the healthcare industry. The “SafeScope Caps” are small and bag-like with an elastic opening. The caps are made completely of latex or vinyl. The caps fit directly over the end of any stethoscope (bell or flat shaped), which is the portion that comes in direct contact with the patient. The caps are disposable, and do not interfere with patient assessment. The “SafeScope Cap” creates a barrier between the patient and stethoscope and helps prevent the likelihood of spreading harmful microorganisms from one patient to another, which may lead to the development of nosocomial infections.

### CROSS-REFERENCE TO RELATED APPLICATIONS

There are no cross-references to any other patents.

### STATEMENT REGARDING FEDERALLY SPONSORED RESEARCH OR DEVELOPMENT

This invention has not been federally sponsored nor has there been any federal research or development related to this patent application.

### BACKGROUND OF THE INVENTION

This invention pertains to all medical/healthcare settings in which a stethoscope is used to provide aide in medical assessments. The stethoscope is the most common and readily available piece of medical equipment in the healthcare industry. Most healthcare providers either own or have liberal access to a stethoscope. The stethoscope can be a potent source of harmful microorganisms and nosocomial infection generating microbes. One study found that 80% of 200 randomly tested stethoscopes were contaminated with microbes. (Eckler, J; Combating Infection from Nursing Journal 27T(:20, 1997).

Each year an estimated 2 million patients acquire nosocomial infections (hospital acquired). These infections are estimated to cost more than 4.5 billion per year and pg. 1961)

The use of medical asepsis and appropriate barrier precautions to reduce the risk of developing nosocomial infections (hospital-acquired) is essential in caring for patients, especially for immunocompromised patients. Children and the elderly who are ill readily fall into this category. (Wong's Essentials of Pediatric Nursing, Wong/Eaton 6^{th} ed. 2001, pg. 766)

### SUMMARY OF THE INVENTION

The “SafeScope Cap” will aide in the prevention of developing nosocomial infections. By applying the “SafeScope Cap”, a barrier is created between the patient and the healthcare professionals stethoscope. The “SafeScope Cap” is limited to a single use and should be disposed of after each use.

By utilizing the “SafeScope Cap” the risk of patients acquiring harmful microorganisms from stethoscopes and risking developing nosocomial infections is drastically decreased.

### BRIEF DESCRIPTION OF THE SEVERAL VIEWS OF THE DRAWINGS

FIG. 1A

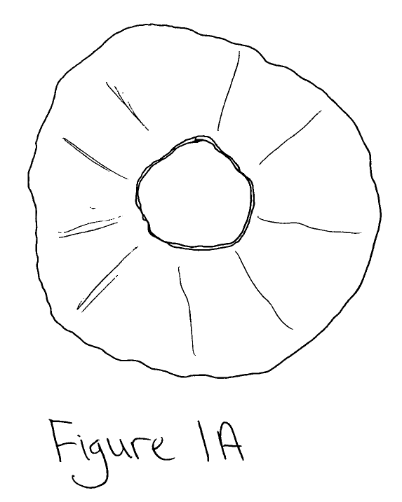

Depicts an aerial or top view of the “SafeScope Cap”. It shows the small elastic opening and the circular body of the appliance.

FIG. 1B

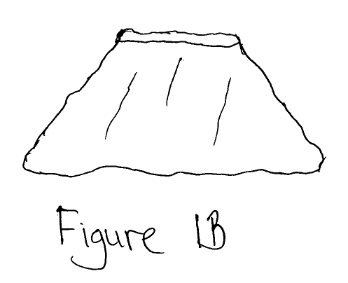

Depicts the side view of the “SafeScope Cap”. It shows how the appliance would look if held by the elastic opening. It shows the small circular top and how the appliance gets wider as you observe the body of it.

FIG. 2A

Depicts the “SafeScope Cap” in practical application. In this drawing the appliance is shown applied to a stethoscope with a bell end.

FIG. 2B

Depicts the “SafeScope Cap” in practical application. In this drawing the appliance is shown applied to a stethoscope with a flat end.

### DETAILED DESCRIPTION OF THE INVENTION

Raw Materials

5 inch piece of round latex/vinyl
12-inch piece of elastic cord (type used for beading, doll clothes, etc.)
small tube of superglue
   Production

Take the 5-inch piece of round latex/vinyl and lay it flat. Take the elastic cord and lay it straight across a portion of the outer edge of the latex/vinyl. Fold the latex/vinyl over the portion of the elastic cord and glue the edge down, leaving the elastic to move freely enclosed in the material. Leave about 1 inch of the elastic cord sticking out and work from the longer end of the cord. Continue to fold a small portion of the outer edge of the material and glue. About halfway through the process the material will begin to take an octagon-like shape, this is exactly what you want. Once you have completely glued all the edges over the elastic (being careful not to glue any of the elastic to the material) you should have a portion of elastic sticking out and very close to each other. Grab the two pieces of elastic and hold the latex/vinyl material in the center. Gently tug at the elastic while holding the material in the center and the material will begin to gather/draw together forming what looks like a cap. Once you pull the material close enough to only get about 2 fingers in the small hole created, tie a double knot with both ends of the cord (make sure the knot is tied as close to being within the material as possible) and cut off the excess cord. The “SafeScope Cap is now ready to be applied to the stethoscope.

Utilization

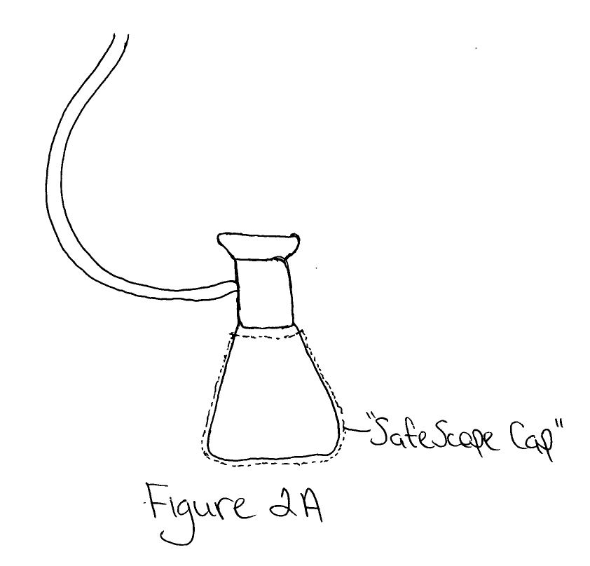

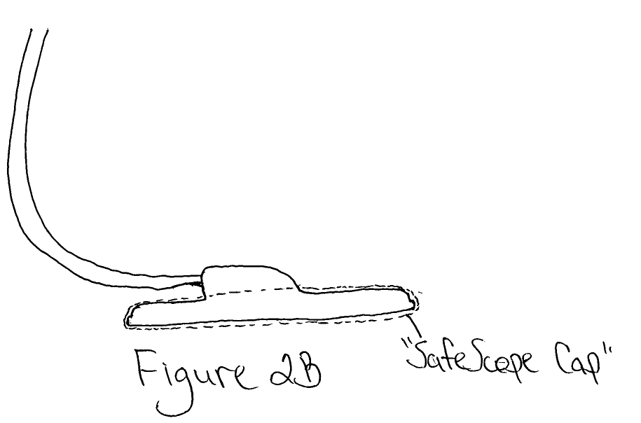

Take the “SafeScope Cap” and spread the elastic opening. Place a portion of the open end onto the bell of a stethoscope. Next work around the bell of the stethoscope until the entire portion of the bell of the stethoscope is covered by the “SafeScope Cap”. (see FIGS. 2A and 2B). The “SafeScope Cap” is now ready for use.

1. “We claim that “safescope caps” to:
not interfere with the delivery of healthcare assessments via stethoscope utilization
not affect patients who have been diagnosed with a “latex sensitivity” by utilizing the vinyl “SafeScope Cap”
be an adequate barrier precaution for infections transmitted by airborne, droplet, and contact routes of transmission
be a barrier of all body fluids, secretions, and excretions in the transmission of nosocomial pathogens
